(19) United States
(12) Patent Application Publication
(10) Pub. No.: US 2008/0182806 A1
(43) Pub. Date: Jul. 31, 2008

(54) USE OF ACETYLATED OR ESTERIFICATED AZACYTIDINE, DECITABINE, OR OTHER NUCLEOSIDE ANALOGS AS ORAL AGENTS FOR THE TREATMENT OF TUMORS OR OTHER DYSPLASTIC SYNDROMES SENSITIVE TO HYPOMETHYLATING AGENTS

(75) Inventor: Giuseppe Pizzorno, Las Vegas, NV (US)

Correspondence Address: DLA PIPER US LLP, 4365 EXECUTIVE DRIVE, SUITE 1100, SAN DIEGO, CA, 92121-2133, US

(73) Assignee: Nevada Cancer Institute, Las Vegas, NV (US)

(21) Appl. No.: 12/020,354

(22) Filed: Jan. 25, 2008

Related U.S. Application Data
(60) Provisional application No. 60/897,765, filed on Jan. 25, 2007.

(51) Int. Cl. A61K 31/70; A61P 7/00

(52) U.S. Cl. 514/42

(57) ABSTRACT

Compositions and methods for treating various diseases and disorders, such as for a myeldoysplastic syndrome are provided. The methods include administering to the subject a therapeutically effective amount of a pharmaceutical composition comprising at least one of an esterificated azacytidine, an acetylated azacitidine, an esterificated decitabine, and an acetylated decitabine, such as 2′,3′,5′-triacetyl-5-azacytidine.

### CROSS-REFERENCE TO RELATED APPLICATIONS

This application claims priority under 35 U.S.C. §119 (e) to U.S. Patent Application Ser. No. 60/897,765 filed Jan. 25, 2007, which is herein incorporated by reference in its entirety.

### TECHNICAL FIELD

This disclosure relates generally to compositions and methods for the treatment of certain tumors and other dysplastic disorders, and more specifically, to using for such purposed heterocyclic compounds, such as functionalized azacytidine, functionalized decitabine, or other nucleoside analogs.

### BACKGROUND INFORMATION

Myelodysplastic syndromes (MDSs) are a diverse collection of haematological conditions united by ineffective production of blood cells and varying risks of transformation to acute myelogenous leukemia. The main characteristics are peripheral cytopenias and dysplasia of hematopoietic progenitor cells. Although not a true malignant neoplasm, MDS is nevertheless classified within the hematological neoplasms. MDS is thought to arise from mutations in the multi-potent bone marrow stem cell, but the specific defects responsible for these diseases remain poorly understood. Differentiation of blood precursor cells is impaired, and there is a significant increase in levels of cell death (apoptosis) in bone marrow cells. Clonal expansion of the abnormal cells results in the production of cells which have lost the ability to differentiate.

The exact number of people with MDS is not known because the condition can go undiagnosed and there is no mandated tracking of the syndrome. Some estimates are on the order of 10,000 to 20,000 new cases each year in the US alone. The incidence is probably increasing as the age of the population increases.

The goals of therapy are to control symptoms, improve quality of life, improve overall survival, and decrease progression to acute myelogenous leukemia (AML). Treatment options include supportive care, with blood product support and hematopoeitic growth factors (e.g. erythropoietin) for low-risk patients to stem-cell transplantation for young patients.

Epigenetic modulation of gene function through DNA methylation has been shown to silence suppressor genes and increase the risk for AML transformation. A nucleoside analog with DNA hypomethylating activity 5-azacytidine (Vidaza) and its derivative 5-aza-2-deoxycytidine/decitabine (Dacogen) have been recently approved by the FDA for MDS treatment. They appear to reduce hypermethylation and induce re-expression of key tumor suppressor genes in MDS. Compared to supportive care, both agents show an overall response (60% vs. 5%) and a longer time to progression to AML or death and improvement of quality of life but with limited overall survival advantage. Recent studies have indicated that lower doses and longer administration of DNA methylation inhibitors may be more efficacious than previously studied higher dosing regimens.

A Phase III trial investigated the effect of 75 mg/m² of azacytidine administered subcutaneously daily for 7 days repeated every 4 weeks versus best supportive care. A 60% response rate was achieved in lower-risk patients with refractory anemia and 61% response in the higher-risk groups. Interestingly, the mean time to response was six treatment cycles, indicating the importance of longer term administration. In a Phase II trial, decitabine was given at 15 mg/m² intravenously over 4 hours three times a day for 3 days with an overall response rate of 49%.

Considering the short half-life of both drugs in serum and the need for long term administration, it is clear that alternative routes of administration or new oral agents or formulations are required to facilitate more flexible regimens and improve patients' quality of life.

The poor absorption of azacitidine when administered orally, coupled with its instability in aqueous solutions and in biologic fluids, due to rapid hydrolysis to byproducts including 5-azacytosine and 5-azauracil, partially due to its breakdown as the result of bacterial action in the large intestine, precludes the use of azacitidine in oral formulation. Accordingly, it is desirable to have better compositions to serve the above-described needs. We provide such compositions.

### SUMMARY

Studies in the past on similar molecules have indicated that the esterification of the free hydroxyl groups on the ribose moiety to generate 2′,3′,5′-triacetylderivatives lead to prodrugs that are rapidly absorbed orally without formation of major metabolites in the gastro-intestinal tract.

Accordingly, in one embodiment, a pharmaceutical composition is provided, the composition comprising at least one compound selected from the group consisting of an esterificated azacytidine, and an esterificated decitabine. One example of such a compound is 2′,3′,5′-triacetyl-5-azacytidine.

In other embodiments, various prodrugs comprising the same compounds are provided, as well as methods of treating a variety of disorders and diseases, such as myelodysplastic syndrome, using the same.

### BRIEF DESCRIPTION OF THE DRAWINGS

FIG. 1 shows chromatograms characterizing some compounds of the present invention.

FIG. 2 presents results of in vivo studies some compounds of the present invention.

FIG. 3 presents results of in vivo studies some compounds of the present invention.

FIG. 4 presents data characterizing anti-tumor activity of compounds of the present invention.

### DETAILED DESCRIPTION

The following definitions are used below, unless otherwise described:

The term “azacytidine,” also known as “5-azacytidine” refers to a compound that is a pyrimidine nucleoside analog of cytidine having antineoplastic activity. Proper chemical names of azacytidine include 4-amino-1-β-D-ribofuranosyl-1,3,5-triazin-2(1H)-one or 4-amino-1-[3,4-dihydroxy-5-(hydroxymethyl)oxolan-2-yl]-1,3,5-triazin-2-one, and the chemical formula of azacytidine (without specifying the stereochemistry thereof) is:

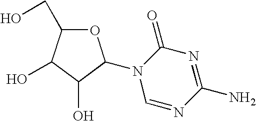

One example of a particular stereochemical configuration in which azacytidine may exist may be illustrated as shown by the formula:

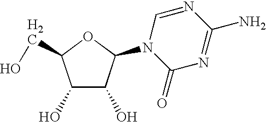

The term “decitabine,” also known as “5-aza-2′-deoxycytidine” refers to a compound that is a cytidine antimetabolite analog with potential antineoplastic activity, having the proper chemical name 4-amino-1-(2-deoxy-b-D-erythro-pentofuranosyl)-1,3,5-triazin-2(1H)-one, and having the formula:

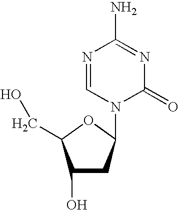

The term “functionalized” refers to a compound that has been modified to have a particular chemical group or moiety, which group or moiety was absent prior to functionalization.

The term “esterificated” refers to a compound having at least one ester moiety as a result of esterification, when such ester moiety was absent prior to the reaction of esterification.

The term “esterification” refers to a chemical reaction in which two functional groups, commonly an alcohol and an acid group, react to form an ester as the reaction product.

The term “ester” refers to an organic molecule having at least one ester group with the general structure —(C═O)—O—.

The term “acetylated” refers to an organic compound having at least one acetyl functional group as a result of acetylation, when such ester moiety was absent prior to the reaction of acetylation.

The term “acetyl” refers to the acyl of acetic acid, having the chemical structure —(C═O)—O—CH₃.

As used herein, the term “substituted” is contemplated to include all permissible substituents of organic compounds. In one example, the permissible substituents can include acyclic and cyclic, branched and unbranched, carbocyclic and heterocyclic, and aromatic and nonaromatic substituents of compounds. The permissible substituents can be one or more and the same or different for appropriate compounds. For the purposes of this disclosure, the heteroatoms, such as nitrogen, can have hydrogen substituents and/or any permissible substituents of organic or inorganic compounds described herein which satisfy the valences of the heteroatoms. This disclosure is not intended to be limited in any manner by the permissible substituents of compounds.

The terms “optional” or “optionally” refer to occurrence or non-occurrence of the subsequently described event or circumstance, and that the description includes instances where said event or circumstance occurs and instances where it does not. For example, in such context, the sentence “optionally substituted alkyl group” means that the alkyl group may or may not be substituted and the description includes both a substituted and an unsubstituted alkyl group.

The term “effective amount” of a compound refers a non-toxic but sufficient amount of the compound that provides a desired effect. This amount may vary from subject to subject, depending on the species, age, and physical condition of the subject, the severity of the disease that is being treated, the particular compound used, its mode of administration, and the like. Therefore, it is difficult to generalize an exact “effective amount,” yet, a suitable effective amount may be determined by one of ordinary skill in the art.

The term “pharmaceutically acceptable” refers to a compound, additive or composition that is not biologically or otherwise undesirable. For example, the additive or composition may be administered to a subject along with a compound of the invention without causing any undesirable biological effects or interacting in an undesirable manner with any of the other components of the pharmaceutical composition in which it is contained.

The term “prodrug” refers to an agent that is converted into the parent drug in vivo. Prodrugs may, for instance, be bioavailable by oral administration whereas the parent drug is not. The prodrug may also have improved solubility in pharmaceutical compositions over the parent drug. A prodrug may be converted into the parent drug by various mechanisms, including enzymatic processes and metabolic hydrolysis.

The term “carrier” refers to a material added to a chemical or formulation to facilitate its preparation, storage or use.

The term “excipient” refers to a medicinally inactive component contained in a drug formulation, including, for example, bulking agents, stabilizing agents, preservatives, salts, or solvents.

The term “stabilizer” refers to a substance added to prevent a possible undesirable change in state of another substance.

As used herein, the term “patient” refers to organisms to be treated by the methods of the present invention. Such organisms include, but are not limited to, humans. In the context of the invention, the term “subject” generally refers to an individual who will receive or who has received treatment for the treatment of a disease, disorder or pathology.

References in the specification and concluding claims to parts by weight or mass of a particular component in a composition denotes the weight or mass relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight or mass percent (wt. % or mass %) of a component, unless specifically stated to the contrary, is based on the total weight of the formulation or composition in which the component is included.

According to embodiments of the present invention, functionalized azacytidine or decitabine may be orally administered as prodrugs for the treatment of myelodysplastic syndromes or other neoplastic malignancies sensitive to DNA methyltransferases inhibitors to a patient suffering from such disorders, syndromes, or malignancies.

Such functionalized derivatives of azacytidine or decitabine include their respective esters, and optionally substituted derivatives or analogs thereof, wherein the ester groups in such esters are formed using the free hydroxyl groups of azacytidine or decitabine.

One example of such an ester is acetylated azacytidine or decitabine, such as 2′,3′,5′-triacetyl-5-azacytidine having the formula I:

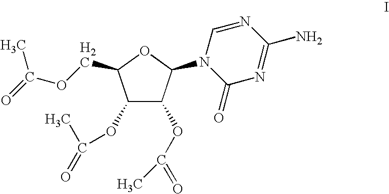

Another example of an ester that may be used is 3′,5′-diacetyl-5-aza-2′-deoxycytidine (which is the acetyl derivative of decitabine).

Acetylated (acylated) derivatives of pyrimidine nucleosides are synthesized by reacting a pyrimidine nucleoside with an activated carboxylic acid. Activated carboxylic acids when treated with appropriate reagents present a carboxylate carbon more susceptible to nucleophilic attack than the original carboxylic acid, for example acid chlorides, acid anhydrides or n-hydroxysuccinimide.

In the preparation of acetyl derivatives of azacitidine or decitabine, because of the presence of group sensitive to esterification, e.g., hydroxyl or amino groups, these groups can be blocked with protecting groups, e.g., t-butyldimethylsilyl ethers or t-BOC groups, respectively, before preparation of the anhydride. With acids containing more than one carboxylate group (e.g., succinic, fumaric, or adipic acid) the acid anhydride of the desired dicarboxylic acid is reacted with a pyrimidine nucleoside in pyridine or pyridine plus dimethylformamide or dimethylacetamide.

Carbyloxycarbonyl derivatives of pyrimidine nucleosides are prepared by reacting the nucleoside with the appropriate carbylchloroformate in a solvent such as pyridine or pyridine plus dimethylformamide under anhydrous conditions.

The compounds of the present invention may be used for the treatment of various disorders, diseases, and pathologies, such as cancer. Accordingly, the compounds of the present invention may be used for preparing pharmaceutical compositions, e.g., by combining these compounds and pharmaceutically acceptable carriers, excipients, and/or stabilizer.

The above-described pharmaceutical compositions may be administered to a mammalian host, such as a human patient in a variety of forms adapted to the chosen route of administration. One type of administration that may be used is oral administration. Other kinds of administration may be also used, if desired, for example, parenteral, intravenous, intraperitoneal, intramuscular, intrathecal, topical or subcutaneous administration.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the present invention can be determined by comparing their in vitro activity, and in vivo activity in animal models. Methods for the extrapolation of effective dosages in mice, and other animals, to humans are known to those having ordinary skill in the art.

Generally, the concentration of the compounds of the present invention in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the compounds of the present invention required for use in treatment will vary not only with the particular salt selected but also with the route of administration, the nature of the condition being treated and the age and condition of the patient and will be ultimately at the discretion of the attendant physician or clinician.

In general, however, a suitable dose can be in the range of between about 0.5 and 100 mg/kg, e.g., between about 10 and 75 mg/kg of body weight per day, such as between about 15 and 60 mg/kg/day. The compounds of the present invention can be conveniently administered in unit dosage form; for example, containing 5 to 1000 mg, such as 10 to 750 mg, for example, 50 to 500 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Optionally, the compositions of the present invention can be administered to a patient in need thereof in combination with other therapeutically beneficial agent(s), to be determined by those having ordinary skill in the art. Various factors that will be taken into account when determining whether such additional therapeutically beneficial agent(s) will be used include consideration of the type of disorder being treated.

The types of disorders, diseases, and pathologies that may be treated using the pharmaceutical compositions comprising compounds of the present invention include cancer, as mentioned above. If the pharmaceutical compositions are used for the treatment of cancer, the kinds of cancer that can be so treated include, for example, hematopoietic cancers, including myelodysplastic syndrome.

### EXAMPLES

The following examples are provided to further illustrate the advantages and features of our processes and systems, but are not intended to limit the scope of this disclosure.

### Example 1

### Analytical Methods in General

Azacytidine was quantitated in plasma using a described and validated LC/MS/MS method. Briefly, plasma samples (200 μL) were extracted using acetonitrile then cleaned up by Oasis MCX ion exchange solid-phase extraction cartridges (Waters Corp, Milford, Mass.). 5-AC was separated on a C₁₈ reverse phase column with gradient elution of ammonium acetate (2 mM) with 0.1% formic acid and methanol mobile phase. Due to the instability of 5-AC in plasma, all processing and handling of 5-AC samples were performed on ice until the samples were dried and reconstituted. Identification was through positive-ion mode and multiple reaction monitoring mode at m/z+244.9→113.0 and 242.0→126.0 for 5-AC and the internal standard, 5-methyl-2′-deoxycytidine, respectively. Zhao, et al., J. Chromatogr. B Analyt. Technol. Biomed. Life Sci .813:81-8, 2004.

Decitabine, its metabolites and the internal standard are detected in plasma through separation on a 250×2.1 mm C₁₈ reverse phase column coupled to a 2 μm precolumn. The mobile phase consisted of 5% acetonitrile in 10 mM ammonium formate at a flow rate of 0.2 mL/min. Pure acetonitrile was then added to the flow at 0.2 mL/min via a separate HPLC pump and mixed post-column prior to the entrance to the ion source. The LC eluate was introduced into the API source at 20 μL/min after a 95:5 (LC/MS) split. The mass spectrometer was operated under electrospray ionization (ESI) with an ion-spray voltage of +4700 V. A dwell time of 600 ms and a pause time of 5 ms between scans were used to monitor the precursor/product ion pairs at m/z 229/113 for decitabine and m/z 247/115 for 5,6-dihydro-5-azacytidine (internal standard). Liu, et al., Rapid Comm. Mass Spec., 20:1117-1126, 2006.

### Example 2

### Sample Preparation

The stock solutions of azacitidine and decitabine were prepared by dissolving the accurately weighed drugs in 10 mL of methanol to a final concentration of 1 mg/mL and stored in glass vials at −80 C. Working solutions were prepared fresh daily by diluting the stock solution with methanol. Microliter volumes of azacitidine or decitabine working solution were added into plasma to prepare calibration standards. A 10 μL aliquot of the stock solution of the internal standards (100 μg/mL) was diluted to 1000 μL with water as the working solution. Plasma samples were loaded on an Oasis MCX SPE cartridge, which had been pre-activated and equilibrated with 1.0 mL of methanol and 1.0 mL of 0.1 N HCl, respectively.

The column was then eluted in sequence with 1.0 mL 0.1 N HCl, 1.0 mL water, 1.0 mL 2.0% methanol, 1.0 mL 50% methanol, 1.0 mL methanol, and 1.0 mL 2.0% NH₄OH in 98% methanol. The NH₄OH/methanol fraction was collected and the solvent evaporated under a stream of nitrogen. All these steps were carried out in a refrigerator at 4° C. This step was necessary to prevent degradation of azacitidine or decitabine and to increase its extraction recovery. The residue was reconstituted in 200 μL 4 C water and analyzed immediately by LC/MS.

### Example 3

### Preparation of 2′,3′,5′-triacetyl-azacytidine (TAC) (Compound I)

First, trimethylsilylated 5-azacytosine (intermediate 1) was prepared according to the reaction scheme A:

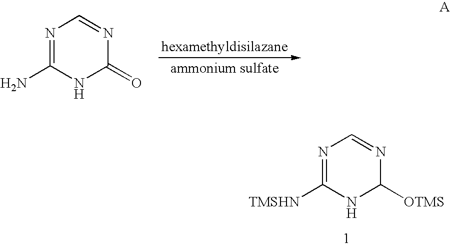

In a 150 mL, 3-necked flask, a mixture of 5-azacytosine (10 g, 119 mmol), hexamethyldisilazane (50 mL) and ammonium sulfate (0.2 g) was heated at reflux for 2 hours. A fresh amount of ammonium sulfate (0.1 g) was added, and the reflux was continued for 6 hours longer. The initial slurry turned into a clear, pale-yellow, solution and no more gas evolved at the end of the reflex. The excess hexamethyldisilazane was removed under reduced vacuum to afford trimethylsilylated 5-azacytosine (1) as an off-white residue, which was used in next step without further purification.

Next, 1,2,3,5-tetra-O-acetyl-β-D-ribofuranose (intermediate 2) was prepared according to the reaction scheme B:

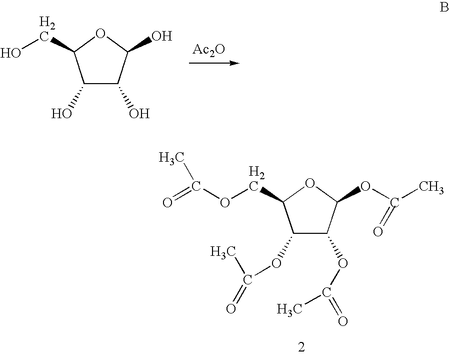

The intermediate 2 was prepared according to the method described in the following reference: Johnson et al. “Chemistry of the vitamin B12 group. II. Synthesis of 5,6-dimethyl-1-α-D ribofuranosylbenzimidazole” J. Chem. Soc. 1953, 3061-6. The intermediate 2 was characterized as follows.

¹H NMR(CDCl₃, 300 MHZ): δ6.12 (s, 1H), 5.30-5.31(m, 1H), 5.30(s, 1H), 4.33-4.36(m, 1H), 4.30(dd, 1H, J=3.3 Hz, 14.4 Hz), 4.11(dd, 1H, J=5.4 Hz, 11.7 Hz), 2.09(s, 3H), 2.06(s, 3H), 2.05(s, 3H), 2.04(S, 3H) ppm; ¹³C NMR (CDCl₃, 75 MHz): δ 170.0, 169.2, 168.0, 168.5, 97.7, 78.8, 73.6, 70.0, 63.2, 20.6, 20.2, 20.0, 20.0 ppm.

The title compound, i.e., 2′,3′,5′-tricetyl-azacytidine, was then prepared using intermediates 1 and 2, according to the reaction scheme C:

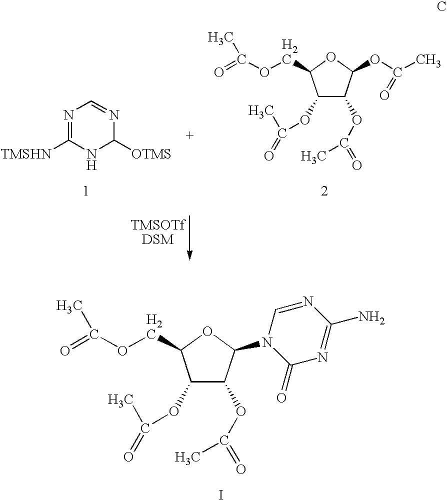

Trimethylsilylated 5-azacytosine 1 (10 g, 39 mmol) and 1,2,3,5-tetra-O-acetyl-β-D-ribofuranose 2 (11.7 g, 36.8 mmol) were dissolved in dichloromethane (84 mL), and the mixture was cooled at 0° C. with ice-water bath. TMSOTf was added slowly at this temperature, then stirred at room temperature for 3 hours. The reaction mixture was poured into a solution of Na₂CO₃ (4.3 g) and NaHCO₃ (4.3 g) in H₂O (60 mL) and ice (35 g). The mixture was then extracted with dichloromethane (3×100 mL). The combined organic layer was washed with cold H₂O (70 mL), cold brine (70 mL), dried over anhydrous Na₂SO4. The residue, after removal of the solvent, was recrystallized from a mixture of dichloromethane and hexane to provide the desired compound I (11.6 g, 85% yield). The title product I was characterized as follows.

¹H NMR (CDCl₃, 500 MHz): δ 8.18(s, 1H), 7.69(s, 1H), 6.33(s, 1H), 5.82(d, 1H, J=3.0 Hz), 5.54(t, 1H, J=4.0 Hz), 5.41(t, 1H, J=6.0 Hz), 4.30-4.41(m, 3H), 2.11(s, 3H), 2.10(s, 3H), 2.08(s, 3H) ppm. ¹³C NMR (CDCl₃, 125 MHz): δ 170.3, 169.6, 169.5, 166.0, 156.0, 153.1, 89.7, 79.9, 73.7, 69.9, 62.8, 20.7, 20.4, 20.3 ppm.

The title product and its active metabolite azacytidine (ACT) were then subjected to LC/MS/MS analysis using the following protocol. Stock solutions were prepared by weighing out 5-10 mg of TAC which was dissolves in methanol to a final concentration of about 0.5 mg/ml. The solution was labeled as “TAC Stock.” 2-3 mg of ACT were then weighed and dissolved in methanol to a final concentration of about 0.2 mg/ml. The solution was labeled as “ACT Stock.” Finally, the TAC and ACT stocks were diluted to a concentration of about 50 μg/ml in methanol, and labeled as “ACT/TAC Stock.” All solutions were stored at −20° C. ACT/TAC Stock was diluted 10 timed with AcNi to be used as working Standards and controls.

Internal standard was then prepared as follows. 2-3 mg of ADT were weighed and dissolved in methanol to a final concentration of about 0.2 mg/ml, followed by dilution to about 10 μg/ml in methanol and labeling as “ADT IS Stock,” which was then diluted 50 times in AcNi to a concentration of about 200 ng/ml, and labeled as “Working IS.” This represents about 20 ng for a 100 μl sample, which represents about 200 ng/ml for a 100 μl plasma sample.

Extraction was then performed as follows. Preparation of standards and controls (i.e., initial preparation) included adding 100 μl of each standard and control to a 1.5 ml Eppendorf tube. 100 μl of the ADT IS Stock was added to each of the above as well as an Internal Standard only tube. All were diluted to 400 μl with AcNi.

100 μl of pooled negative plasma was added to all tubes, vortexed for 10 seconds at 3,000 rpm, and centrifuged at 6,000 rpm (2,600 g) for 10 minutes, followed by decanting supernatant into a 10×75 mm disposable glass test tube, evaporating liquid down to approximately 50 μl under nitrogen at 37° C., diluting with 1 ml 2% phosphoric acid, and vortexing for 10 seconds at 3,000 rpm.

Samples were then initially prepared as follows. A measured aliquot of the acetonitrile extracted plasma sample was removed and placed into a 10×75 mm disposable glass test tube, followed by evaporating liquid down to approximately 50 μl under nitrogen at 37° C., diluting with 1 ml 2% phosphoric acid, and vortexing for 10 seconds at 3,000 rpm.

The samples were then analyzed by the SPE, HPLC and MS/MS methods. In the SPE method, Bond Elute PLEXA PCX 1 ml columns were labeled, placed on vacuum manifold, and conditioned with 1 ml of methanol followed by 1 ml of de-ionized water. 1 ml of 2% phosphoric acid was added and elution was stopped. Standards, controls and samples were added and columns were allowed to drip by gravity. Maximum flow rate was about 0.5 ml/min.

Washing was the performed using 1 ml of 2% phosphoric acid, 1 ml of de-ionized water, and 1 ml of methanol/AcNi (1/1), followed by eluting into 10×75 mm glass test tubes containing 200 μl of 25% formic acid in methanol, and twice with 500 μl of 2% AmOH in MeOH/AcNi (1/1), and evaporating to dryness with nitrogen at 37° C. 100 μl of Mobile Phase A was added, vortexed for 10 seconds at 3,000 rpm, and transferred to LC autosampler vials. The auto sampler was placed immediately at 4° C.

In the HPLC method, the following parameters were used. Shimadzu SIL-HTc controller with 2 LC-20AD pumps and DGU-20A3 degasser was used. The column was Varian Pursuit C18, 3 micron particle size; 100 mm long by 2.0 mm diameter. 0.1% Formic Acid was Mobile Phase A, and 0.1% formic Acid, 90% methanol was Mobile Phase B. Flow rate was 300 μl/min and column temperature was 40° C. Injection volume was 20 μl.

The following time program was utilized: 0.0, 2%; 2.0, 2%; 5.5, 90%; 8.0, 90%; 8.5, 2%; 10.0, stop.

In the MS/MS method, Applied Biosystems API 3200 apparatus was utilized, the ion source was Turbo V Ion Spray, polarity was for Positive Ion, and other parameters are shown below:

| | Parent | Product | | | | | | |
|---|---|---|---|---|---|---|---|---|
| | Ion, | Ion, | Dwell, | | | | | |
| Analyte | msec | msec | msec | DP | CEP | CXP | EP | CP |
| ACT | 245.1 | 113.1 | 200 | 11 | 12 | 4 | 5 | 17 |
| ADT | 229.1 | 112.8 | 200 | 16 | 14 | 6 | 5 | 17 |
| TAC | 371 | 139.1 | 100 | 11 | 20.5 | 4 | 5 | 17 |

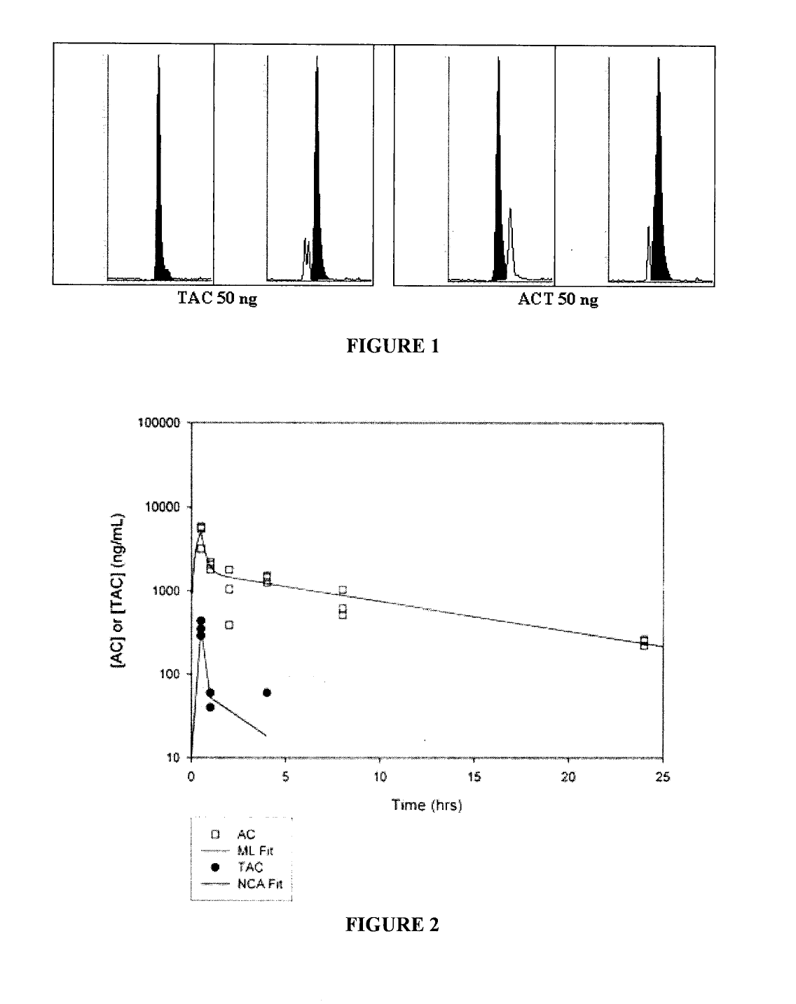

Curtain gas was supplied at 25 ml/min, ion voltage was 5,000 V, source temperature was 500° C., gases #1 and #2 were supplied, respectively, at 50 ml/min and 60 ml/min, source heater was on, CAD gas was at 5 mTorr, resolution Q1 was low, and resolution Q2 was unit. FIG. 1 shows representative chromatograms of TAC and ACT in the presence of the Internal Standard Deoxy-azacytidine.

### Example 4

### Pharmacokinetic Analysis

Individual concentration-time data were analyzed using noncompartmental methods using WinNonlin Professional (Pharsight, Mountain View, Calif.). Maximum plasma concentration (Cₘₐₓ) is the observed value, as is the time to Cₘₐₓ (Tₘₐₓ). The terminal rate constant (λ_{z}) is determined from the slope of the terminal phase of the plasma concentration-time curve using uniform weight. The terminal half-life (T_{1/2}) was calculated as 0.693 divided by λ_{z}. Area under the concentration-time curve (AUC) was calculated using the log/linear trapezoidal rule.

The AUC was extrapolated to infinity (AUC_{0-∞}) by using the equation, AUC_{0-∞}=AUC₀₋ₜ+Cₗₐₛₜ/λ_{Z}. where Cₗₐₛₜ was the final quantifiable concentration. The percent extrapolated was determined using the equation=(AUC_{0-∞}−AUC₀₋ₜ)/AUC_{0-∞}×100%). Dose-normalized AUC_{0-∞} is calculated by dividing AUC_{0-∞} by the nominal dose administered. Apparent systemic clearance (Cl/F) was calculated by dividing the dose by AUC_{0-∞}. Apparent volume of distribution (Vd/F) was calculated by dividing apparent systemic clearance by the terminal rate constant.

Pharmacokinetic parameters are summarized using descriptive statistics. Graphical presentation of concentration-time profiles consisted of the average and standard deviation of the 5-AC concentration determined at each time point. Dose-independent pharmacokinetic parameters (Tₘₐₓ, T_{1/2}, AUC percent extrapolated, Cl/F, and Vd/F) were compared using a Student's t test between the various pharmacokinetic periods. One-way analysis of variance (ANOVA) was used to compare the differences in clearance and dose-normalized AUC_{0-∞} as a function of dose level. ANOVA and Student's t test are performed using JMP Statistical Discovery software (version 4.0.4; SAS Institute, Cary, N.C.). The a priori level of significance was set at P<0.05.

### Example 5

### Animal Experiments

Plasma azacitidine levels after administration of 2′,3′,5′-triacetyl-azacitidine were determined in mice at various times (15 minutes, 30 minutes, 1 hour, 2 hours, 4 hours and 6 hours) after administration of the acetyl derivatives of azacitidine. Groups of mice (n=3 per time point) received oral administration of 2′,3′,5′-triacetyl-azacitidine (300 mg/kg); this dose of the acyl derivatives of azacitidine is the molar equivalent of 200 mg/kg azacitidine. At the appropriate time points, blood samples (400 μl) were taken from mice via the retro-orbital sinus, immediately centrifuged, extracted and analyzed as described above.

Bioavailability of 2′,3′,5′-triacetyl-azacitidine (azacitidine) was determined as follows. Equal doses of 2′, 3′, 5′-triacetyl-azacitidine (300 mg/kg) were administered to two groups of mice: group A by oral administration and group B by intraperitoneal injection. Plasma azacitidine levels were determined in mice at various times (15 minutes, 30 minutes, 1 hour, 2 hours, 4 hours and 6 hours) after administration of the acetyl derivatives of azacitidine.

Exposure to azacitidine was evaluated following administration of equal molar doses of 2′,3′,5′-triacetyl-azacitidine orally or azacitidine by intraperitoneal injection. A 300 mg/kg dose of 2′,3′,5′-triacetyl-azacitidine was administered by oral gavage to a group of mice and the AUC of the resulting azacitidine and compared to the AUC obtained in a similar group of mice receiving the equal molar dose of azacitidine (200 mg/kg) administered intraperitoneally as a bolus. Plasma azacitidine levels were determined at various times (15 minutes, 30 minutes, 1 hour, 2 hours, 4 hours and 6 hours) after administration of the azacitidine derivatives.

A similar set of experiments was conducted to evaluate the pharmacokinetic properties of 3′,5′-diacetyl-5-aza-2′-deoxycytidine as compared to the parent compound decitabine.

Pharmacokinetic analysis was further conducted to evaluate pharmacokinetics and metabolism of TAC in mice. C57/BL6 female mice of ˜20 g body weight were dosed with 38 mg/kg TAC or 25 mg/kg ACT. C57BL/6 X DBA/2 F1(BDF1) female mice, weighing 18 to 20 g, wereobtained from Charles River. The mice were housed in a constant-temperature facility with a 6 a.m. to 6 p.m. photoperiod and were given standard laboratory chow and water ad libitum. TAC was administered p.o. while ACT was administered I.V via tail vein injection or p.o. TAC and ACT were solubilized in PBS immediately before administration.

Lymphoid leukemia L1210 was carried in BDF1 mice by weekly i.p. passages. Ascites fluid was aspirated from a donor mouse and sampled, and an aliquot was carefully diluted and counted in a hemocytometer. The original suspension of cells was then diluted appropriately in RPMI 1610 medium to 1×106 cells/ml. and injected i.p. (1×105 cells/0.1 ml/mouse) into recipient BDF1 mice.

A daily oral administration injection of TAC (38 mg/kg) diluted in PBS was given to leukemic BDF1 mice by oral gavage starting 24 hours from the inoculation of L1210 cells for a total of 5 days. A group of untreated leukemic mice of the same strain, sex, age, and weight served as controls.

Mice were dosed (0.01 mL/g fasted body weight) by tail vein injection or by oral gavage. Blood samples (three per time point) were obtained by ocular bleeding using a Natelson pipette at 10, 15, 30, 60, 120, 240, 480, 720 and 1,440 min after dosing. Blood was collected by ocular bleeding and centrifuged for 4 min at 13,000×g to obtain plasma. The aspirated plasma was immediately extracted with acetonitrile and stored at −70° C. until analysis LC/MS analysis.

The pharmacodynamic effect of TAC and ACT was determined at 24 and 48 hours following drug administration after the female C57/BL6 mice were euthanized, their tissues rapidly harvested, snap-frozen in liquid nitrogen, and stored at −80° C. The effect on methylation was determined by using the Epigentek Methylamp™ Global DNA Methylation Quantification Kit. Isolated genomic DNA from mouse tissues 24 or 48 hours after treatment with 25 mg/kg ACT i.v. and 38 mg/kg TAC p.o., methylation status was determined using 100 ng of each gDNA sample.

FIG. 2 illustrates TAC and derived AC C-t profile in non-tumor bearing mice after TAC p.o. dosing. FIG. 2 shows the presence of TAC and its active metabolite ACT in plasma within 15 minutes from the oral administration of the nucleoside pro-drug. It appears that TAC is rapidly deacetylated leading to a minimal accumulation of the prodrug that appears to be below the limit of detection (30 ng/ml) by 4 hours after oral administration of 38 mg/kg (equivalent to 25 mg/kg of ACT). TAC-derived azacytidine is present already after 15 minutes from dosage reaching a peak concentration of approximately 5,000 ng/ml (˜20 uM) at 30 minutes with a pharmacologically relevant concentration of 0.5 uM after 24 hours.

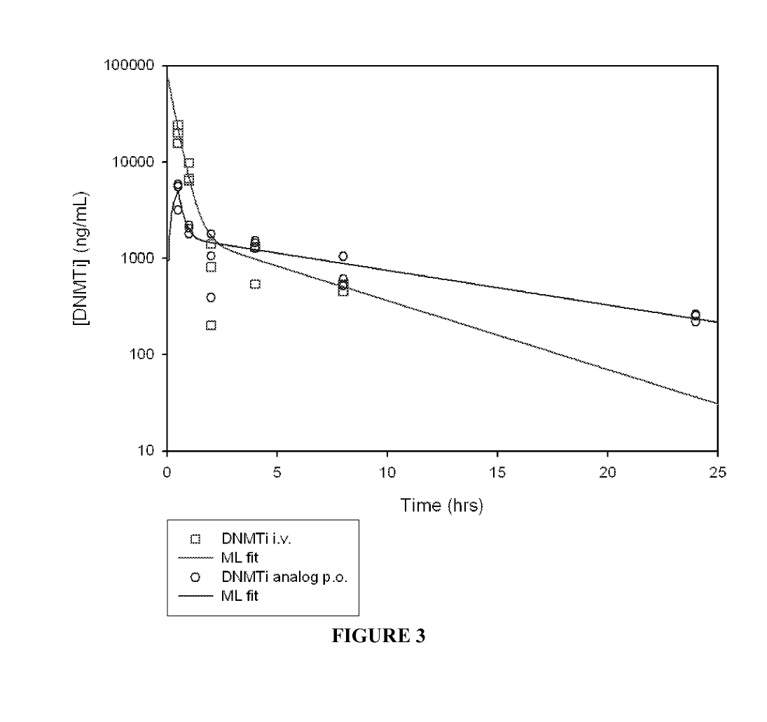

The half-life of the TAC-derived azacytidine is 8.5 hours versus a half-life of 4-5 hours for azacytidine when administered i.v. to indicate a protracted absorption of the nucleoside pro-drug at the gastro-intestinal level, as can be seen from FIG. 3 demonstrating DNMTi plasma C-t profile in non-tumor bearing mice.

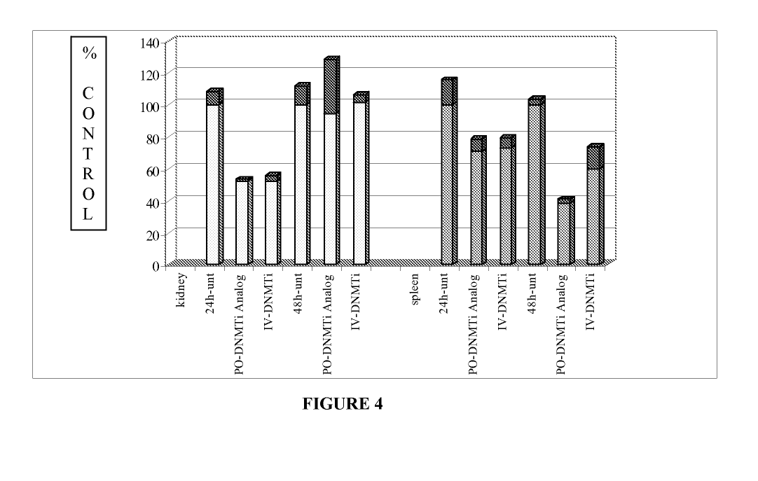

From a pharmacodynamic stand point the effect of TAC derived ACT and i.v. ACT is shown in FIG. 4. A similar 50% reduction in global DNA methylation in the kidneys within 24 hours from the administration of the oral pro-drug and i.v. ACT was observed, the effect subsided after 48 hours. In spleen an initial reduction in methylation was 25-30% for both dosages, with a further decrease to 40% compared to untreated tissue for the oral TAC. The median survival time for the untreated control group was 9 days while the median survival time for the TAC treated group was 14 days resulting in a 55% increased lifespan in the presence of the oral nucleoside prodrug.

Although methods, compositions and devices of the present invention have been described with reference to the above-discussed embodiments and examples, it will be understood that modifications and variations are encompassed within the spirit and scope of the disclosure as defined in the appended claims.

What is claimed is:

1. A pharmaceutical composition comprising:
(a) at least one compound selected from the group consisting of an esterificated azacytidine and an esterificated decitabine; and
(b) a pharmaceutically acceptable carrier, excipient or stabilizer.

2. The pharmaceutical composition of claim 1, wherein the compound is 2′,3′,5′-triacetyl-5-azacytidine.

3. The pharmaceutical composition of claim 2, wherein the compound has the formula I:

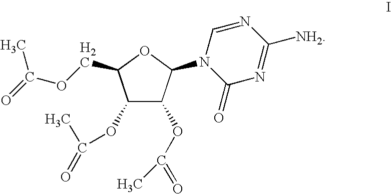

4. The pharmaceutical composition of claim 1, wherein at least one of the esterificated azacytidine or the esterificated decitabine comprises at least one acetyl group.

5. The pharmaceutical composition of claim 1, wherein the compound is the esterificated azacytidine.

6. The pharmaceutical composition of claim 1, wherein the compound is the esterificated decitabine.

7. The pharmaceutical composition of claim 1, wherein the compound is an acetylated azacytidine.

8. The pharmaceutical composition of claim 1, wherein the compound is an acetylated decitabine.

9. A prodrug comprising a compound selected from the group consisting of an esterificated azacitidine, an acetylated azacitidine, an esterificated decitabine, and an acetylated decitabine.

10. The prodrug of claim 9, wherein the compound is 2′,3′,5′-triacetyl-5-azacytidine.

11. The prodrug of claim 10, wherein the compound has the formula I:

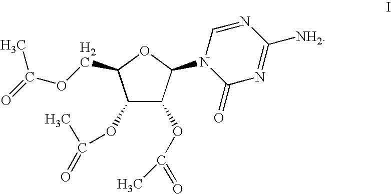

12. The prodrug of claim 9, wherein the esterificated azacytidine or the esterificated decitabine comprises at least one acetyl group.

13. The prodrug of claim 9, wherein the compound is the esterificated azacytidine.

14. The prodrug of claim 9, wherein the compound is the esterificated decitabine.

15. The prodrug of claim 9, wherein the compound is an acetylated azacytidine.

16. The prodrug of claim 9, wherein the compound is an acetylated decitabine.

17. A method for treating a myelodysplastic syndrome in a subject in need thereof, comprising administering to the subject a therapeutically effective amount of a pharmaceutical composition comprising at least one of an esterificated azacytidine, an acetylated azacitidine, an esterificated decitabine, and an acetylated decitabine.

18. The method of claim 17, wherein the compound is 2′,3′,5′-triacetyl-5-azacytidine.

19. The method of claim 2, wherein the compound has the formula I:

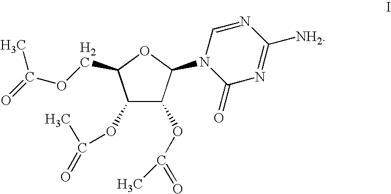

20. The method of claim 17, wherein at least one of the esterificated azacytidine or the esterificated decitabine comprises at least one acetyl group.

21. The method of claim 17, wherein the compound is the esterificated azacytidine.

22. The method of claim 17, wherein the compound is the esterificated decitabine.

23. The method of claim 17, wherein the compound is an acetylated azacytidine.

24. The method of claim 17, wherein the compound is an acetylated decitabine.

25. The method of claim 17, wherein the symptoms of myelodysplastic syndrome are reduced, quality of life is improved, survival is increased and/or progression to acute myelogenous leukemia is reduced.
